SEQUENCE LISTING (18 sequences)

SEQ ID NO: 1 - DNA, 45 bp, Artificial Sequence
  synthetic nucleotide
agtcgtgctgaatgtgtgtgtgtgtgtgtgaccatcgtagcttgc

SEQ ID NO: 2 - DNA, 45 bp, Artificial Sequence
  synthetic nucleotide
gctagctcgagttgatgatgatgatgatgactcggctaagatcga

SEQ ID NO: 3 - DNA, 90 bp, Artificial Sequence
  synthetic nucleotide
aatgatacggcgaccaccgagatctacacaatagcaaacactctttccctacacgacgctcttccgatctgagatttgggtggggacaca

SEQ ID NO: 4 - DNA, 90 bp, Artificial Sequence
  synthetic nucleotide
aatgatacggcgaccaccgagatctacacactcgctaacactctttccctacacgacgctcttccgatcttggtgccaaaaaggttgggg

SEQ ID NO: 5 - DNA, 90 bp, Artificial Sequence
  synthetic nucleotide
aatgatacggcgaccaccgagatctacacaggccttgacactctttccctacacgacgctcttccgatctccactggttggggatcactg

SEQ ID NO: 6 - DNA, 90 bp, Artificial Sequence
  synthetic nucleotide
aatgatacggcgaccaccgagatctacacattgaaggacactctttccctacacgacgctcttccgatctttggcagtaaaatgccaaaa

SEQ ID NO: 7 - DNA, 90 bp, Artificial Sequence
  synthetic nucleotide
aatgatacggcgaccaccgagatctacacccaagagtacactctttccctacacgacgctcttccgatctggtgtgaaaacaccctgctg

SEQ ID NO: 8 - DNA, 90 bp, Artificial Sequence
  synthetic nucleotide
aatgatacggcgaccaccgagatctacaccgatgacgacactctttccctacacgacgctcttccgatctgggggtttcatggcccttta

SEQ ID NO: 9 - DNA, 90 bp, Artificial Sequence
  synthetic nucleotide
aatgatacggcgaccaccgagatctacacctcgaaccacactctttccctacacgacgctcttccgatctattttaaatgcccagcgaag

SEQ ID NO: 10 - DNA, 90 bp, Artificial Sequence
  synthetic nucleotide
aatgatacggcgaccaccgagatctacacgagcatacacactctttccctacacgacgctcttccgatctcgcgttcaaacaaacaaact

SEQ ID NO: 11 - DNA, 90 bp, Artificial Sequence
  synthetic nucleotide
aatgatacggcgaccaccgagatctacactcctgagaacactctttccctacacgacgctcttccgatctattcaccccctctaggcgac

SEQ ID NO: 12 - DNA, 90 bp, Artificial Sequence
  synthetic nucleotide
aatgatacggcgaccaccgagatctacacttcagcagacactctttccctacacgacgctcttccgatctacgcacgggcactcacctag

SEQ ID NO: 13 - DNA, 90 bp, Artificial Sequence
  synthetic nucleotide
aatgatacggcgaccaccgagatctacacgctctgctacactctttccctacacgacgctcttccgatctaattcaacccccccttctta

SEQ ID NO: 14 - DNA, 90 bp, Artificial Sequence
  synthetic nucleotide
aatgatacggcgaccaccgagatctacacgtcgctagacactctttccctacacgacgctcttccgatctttcaycatgaagctttgctt

SEQ ID NO: 15 - DNA, 50 bp, Artificial Sequence
  synthetic nucleotide
acagacatttacagtatacggatgactagcatttagcttagctatcctac

SEQ ID NO: 16 - DNA, 30 bp, Artificial Sequence
  synthetic nucleotide
acagacatttacagtatacggatgactagc

SEQ ID NO: 17 - DNA, 30 bp, Artificial Sequence
  synthetic nucleotide
gtaggatagctaagctaaatgctagtcatc

SEQ ID NO: 18 - DNA, 50 bp, Artificial Sequence; identical to SEQ ID NO: 15
  synthetic nucleotide

(19) United States
(12) Patent Application Publication
(10) Pub. No.: US 2021/0130815 A1
(43) Pub. Date: May 6, 2021

(54) Evaluating Genomic Variation Using Repetitive Nucleic Acid Sequences

(71) Applicant: Florida State University Research Foundation, Inc., Tallahassee, FL (US)

(72) Inventor: Alan R. Lemmon, Tallahassee, FL (US)

(21) Appl. No.: 17/090,454

(22) Filed: Nov. 5, 2020

Related U.S. Application Data
(60) Provisional application No. 62/930,826, filed on Nov. 5, 2019.

(51) Int. Cl. C12N 15/10; C12Q 1/6827; C12Q 1/6853

(52) U.S. Cl. CPC C12N 15/1096; C12Q 2565/519; C12Q 1/6853; C12Q 1/6827

(57) ABSTRACT

Systems and methods for evaluating genomic variation include utilizing tailed primers targeting repetitive genomic regions to amplify multiple regions throughout a genome.

### CROSS REFERENCE TO RELATED APPLICATION

This application claims the benefit of, and priority to, U.S. Provisional Patent Application Ser. No. 62/930,826 filed Nov. 5, 2019, the entirety of which is incorporated by reference and commonly owned.

### SEQUENCE LISTING

The application contains a Sequence Listing electronically submitted via EFS-web to the United States Patent and Trademark Office as a text file named “Sequence_Listing.txt” created Nov. 5, 2020 with a file size of 5 kB. The electronically filed Sequence Listing serves as both the paper copy required by 37 C.F.R. § 1.821(c) and the computer readable file required by 37 C.F.R. § 1.821(c). The information contained in the Sequence Listing is incorporated by reference herein in its entirety.

### FIELD

This disclosure relates to the field of evaluating genomic variation.

### BACKGROUND

Evaluating genomic variation is a critical step in curing human disease, improving crop yields, and understanding the genetics of natural populations. The genomes of many species of interest are large due to repetitive elements, or repeat motifs. Due to their size, genomes are typically subsampled before sequencing in order to make evaluating genomic variation financially feasible. Currently used methods of genome subsampling (i.e. Restriction-Assisted Digest, RAD), however, are highly inefficient because the process employed—selecting DNA fragments by size after cutting with restriction enzymes—is not very repeatable across samples.

The result of this inefficiency is wasted data and high cost. Moreover, existing methods perform worse for species that could benefit the most from genome reduction, namely, species with larger genomes. Lastly, existing methods can only be used to target randomly placed regions in the genome, not regions likely to have important variation, like gene regulatory regions.

### SUMMARY

New systems and methods for subsampling genomes in a flexible way, with high repeatability and efficiency, would be a great benefit to medicine, crop science, and population genetics, among others. The methods and systems disclosed herein are aimed at fulfilling one or more of these needs.

An example of a method for evaluating genomic variation includes (i) generating nucleic acid fragments by fragmenting a nucleic acid, at least one of said nucleic acid fragments having a repeat motif; (ii) ligating an adapter molecule having an adapter sequence to the at least one of said nucleic acid fragments having a repeat motif; and (iii) amplifying at least a portion of the at least one of said nucleic acid fragments having a repeat motif using a tailed primer and an adapter primer, said tailed primer including a first nucleic acid sequence that binds to the repeat motif and a second nucleic acid sequence that does not bind to the at least one of said nucleic acid fragments having a repeat motif, said adapter primer including a nucleic acid sequence homologous to the adapter sequence, thereby producing amplified nucleic acid fragments.

This method may also include one or more of the following features.

The repeat motif may include a nucleotide sequence including at least one of GTⁿ, GTⁿ-H, GTⁿ-HV, GTⁿ-A, V-GTⁿ, HV-GTⁿ, V-GTⁿ-H, HV-GTⁿ-HV, TGⁿ, ACⁿ, CAⁿ, and a reverse complement thereof.

The first nucleic acid sequence may be complementary to the repeat motif.

The second nucleic acid sequence may be at least partially non-complementary to the at least one of said nucleic acid fragments having a repeat motif.

Fragmenting may comprise sonicating the nucleic acid.

The first nucleic acid sequence may be downstream of the second nucleic acid sequence.

A repeat motif may be selected using a bioinformatics protocol comprising (a) loading a nucleic acid sequence into a software program; (b) using a data structure to store a sample of short DNA sequences (“Kmers”) with corresponding melting temperatures (“Tm”); (c) profiling each Kmer for genomic abundance to identify candidates; (d) profiling the candidates for a potential to mis-prime; (e) profiling the candidates for sequence diversity in downstream flank; (f) profiling the candidates for genomic uniformity; (g) profiling the candidates for levels of selection; (h) collapsing similar candidates using degenerate bases; (i) evaluating alignments of flanking regions of the candidates; (j) evaluating the potential for the candidates to be a suitable primer; and (k) selecting at least one suitable repeat motif for use in subsequent steps in the method.

The nucleic acid may comprises DNA.

The adapter primer may include a sequence that is at least partially homologous to the adapter sequence.

An example of a method for simultaneously evaluating genomic variation in first and second species, comprises (i) pooling (a) a first species nucleic acid from the first species, the first species nucleic acid having a first repeat motif and (b) a second species nucleic acid from the second species, the second species nucleic acid having a second repeat motif; (ii) generating nucleic acid fragments by fragmenting the first species nucleic acid and the second species nucleic acid; (iii) ligating an adapter molecule having an adapter sequence to at least one of the nucleic acid fragments; and (iv) amplifying at least a portion of the nucleic acid fragments using a first tailed primer, a second tailed primer, and an adapter primer, the first tailed primer including a first nucleic acid sequence that binds to the first repeat motif and a second nucleic acid sequence that does not bind to at least one of said nucleic acid fragments having the first repeat motif, the second tailed primer including a third nucleic acid sequence that binds to the second repeat motif and a fourth nucleic acid sequence that does not bind to at least one of said nucleic acid fragments having the second repeat motif, the adapter primer including a sequence homologous to the adapter sequence, thereby producing amplified nucleic acid fragments.

This method may include one or more of the following features.

The at least one of the first and second repeat motifs may include a nucleotide sequence including at least one of GTⁿ, GTⁿ-H, GTⁿ-HV, GTⁿ-A, V-GTⁿ, HV-GTⁿ, V-GTⁿ-H, HV-GTⁿ-HV, TGⁿ, ACⁿ, CAⁿ, and a reverse complement thereof.

The first nucleic acid sequence may be complementary to the first repeat motif and the third nucleic acid sequence may be complementary to the second repeat motif.

The second nucleic acid sequence may be non-complementary to the at least one of said nucleic acid fragments having the first repeat motif, and the fourth nucleic acid sequence may be non-complementary to the at least one of said nucleic acid fragments having the second repeat motif.

Fragmenting may comprise sonicating the first species nucleic acid and the second species nucleic acid.

The adapter primer may include a sequence that is at least partially homologous to the adapter sequence.

An example of a system for evaluating genomic variation in a nucleic acid fragment having a repeat motif comprises: (i) a tailed primer including a first nucleic acid sequence that binds to the repeat motif and a second nucleic acid sequence that does not bind to the nucleic acid fragment.

This system may include one or more of the following features.

The system may include an adapter primer having a sequence at least partially homologous to an adapter sequence at an end of the nucleic acid fragment.

The nucleic acid may comprise DNA.

The repeat motif may include a nucleotide sequence including at least one of GTⁿ, GTⁿ-H, GTⁿ-HV, GTⁿ-A, V-GTⁿ, HV-GTⁿ, V-GTⁿ-H, HV-GTⁿ-HV, TGⁿ, ACⁿ, CAⁿ, and a reverse complement thereof.

The second nucleic acid sequence may comprise a P5 adapter sequence and the adapter sequence may comprise a P7 adapter sequence.

A second tailed primer may include a first nucleic acid sequence homologous to the repeat motif and a third nucleic acid sequence that is at least partially non-complementary to the nucleic acid fragment.

The nucleic acid fragment may comprise multiple nucleic acid fragments from divergent species.

Another example of a method for evaluating genomic variation comprises (i) generating nucleic acid fragments by fragmenting a nucleic acid, at least one of said nucleic acid fragments comprising a repeat motif; (ii) ligating an adapter molecule having an adapter sequence to the at least one of said nucleic acid fragments comprising a repeat motif; (iii) amplifying at least a portion of the nucleic acid fragments using a tailed primer and an adapter primer, said tailed primer including a first nucleic acid sequence complementary to the repeat motif and a second nucleic acid sequence that is at least partially non-complementary to the at least one of said nucleic acid fragments comprising a repeat motif, said adapter primer including a sequence at least partially homologous to the adapter sequence, thereby producing amplified nucleic acid fragments.

Another example of a system for evaluating genomic variation in a nucleic acid fragment having a repeat motif has a tailed primer including a first nucleic acid sequence complementary to the repeat motif and a second nucleic acid sequence that is at least partially non-complementary to the nucleic acid fragment.

### BRIEF DESCRIPTION OF THE DRAWINGS

For a fuller understanding of the embodiments disclosed herein, reference is made to the following detailed description, taken in connection with the accompanying drawings illustrating various embodiments of the present disclosure, in which:

FIG. 1 is a flow diagram of an example of a system and method of evaluating genomic variation;

FIG. 2 is a depiction of the steps of an exemplary system and method of evaluating genomic variation;

FIG. 3 is a bar graph obtained from an exemplary system and method depicting the recovery of target regions;

FIG. 4 is a set of graphs showing the effect of annealing temperature on the number of reads mapped to each genomic location using an exemplary system and method;

FIG. 5 is tabulated data showing the effect of annealing temperature and primer concentration on efficiency when a GT¹¹-containing primer is used to enrich human DNA;

FIG. 6 depicts a distribution of read coverage across loci using a GT¹¹-containing primer;

FIG. 7 depicts a correlation of coverage between technical replicates in methods using a GT¹¹-containing primer to enrich human DNA for >75,000 loci in two technical replicates;

FIG. 8 depicts a relationship between sequencing effort and the total number of loci obtained when a GT¹¹-containing primer is used to enrich human DNA; and

FIG. 9 depicts a distribution of GT¹⁰ microsatellites in the human genome.

### DETAILED DESCRIPTION OF EXAMPLE EMBODIMENTS

This disclosure describes systems and methods for evaluating genomic variation, but not all possible examples thereof. Where a particular feature is disclosed in the context of a particular example, that feature can also be used, to the extent possible, in combination with and/or in the context of other examples. The systems and methods may be embodied in many different forms and should not be construed as limited to only the examples and features described here.

Unless defined otherwise, all technical and scientific terms used herein have the same meaning as commonly understood by one of ordinary skill in the art to which the subject matter of this disclosure belongs. Although any methods and materials similar or equivalent to those described herein can be used in the practice or testing of the systems and methods, only certain exemplary methods and materials are described.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, a temperature, and the like, encompasses variations of ±20%, or ±10%, or ±5%, or ±1%, or ±0.1% from the specified value, as such variations are appropriate to prepare the disclosed compositions, use the disclosed systems, and perform the disclosed methods.

“Homologous” nucleic acid sequences include sequences having at least 60%, more preferably at least 70%, more preferably at least 80%, more preferably at least 90%, more preferably 95%, or 100% homology to a nucleic acid or a portion thereof. The homology of one or more sequences may be calculated using conventional algorithms. A homologous nucleic acid sequence may also include a sequence that has less than 60% but more than 30%, such as 50-59%, for example 55%, such as 40-49%, for example 45%, such as 30-39%, for example 35% homology to a nucleic acid sequence.

The systems and methods disclosed herein provide a way to identify and utilize repetitive elements in genomes to enable assessment of genomic variation at less cost and time than conventional methods. Data quality may also be improved in some of the disclosed examples. As described in greater detail below, exemplary data from six model species indicate that the systems and methods disclosed herein are capable of subsampling a diversity of plant and animal genomes with high levels of efficiency and repeatability.

The systems and methods also have several useful advantages over conventional techniques. For example, they may be useful to evaluate genomic variation across individuals of the same or different species to answer questions in population genetics, phylogeography, or phylogenetics. In this context, assessing microsatellite variation, single nucleotide polymorphism (SNP) variation, or DNA sequence variation may be useful.

In another example, the systems and methods may be useful in connection with various agricultural applications. For example, novel or known variations in SNPs or microsatellites can be used to improve yields or quality of plants or animals. The systems and methods may also be used to detect diseased or contaminated individuals.

In another example, the systems and methods may be useful in connection with DNA fingerprinting. In this application, individuals may be identified very accurately and precisely by profiling genomic variation (i.e. patterns of microsatellite lengths). Additional applications include forensics and paternity testing.

In another example, the systems and methods may be useful with direct-to-customer DNA testing. For example, genetic profiles (i.e. microsatellite or SNP profiles) obtained from the disclosed systems and methods may be used to identify ancestry and/or genetic features (i.e. propensity for disease).

In another example, the systems and methods may be useful in connection with food safety and/or fraud analysis. For example, genetic profiles (i.e. microsatellite or SNP profiles) of sampled food may be used to verify the species or variants of a food being sold. A presence of bacteria or other contaminants may also be detected using these profiles.

In another example, the systems and methods may be useful in connection with medicine. For example, bacterial or viral pathogens may be detected using examples of the methods by targeting repeats known to be characteristic of the pathogen (e.g. the Long Terminal Repeat associated with HIV). The DNA in samples taken from patients may be profiled to detect these pathogens. Variants in coding and/or regulatory regions may be assessed to identify propensity for, or presence of, a disease or disorder (e.g. the CAG trinucleotide repeat indicating Huntington's disease).

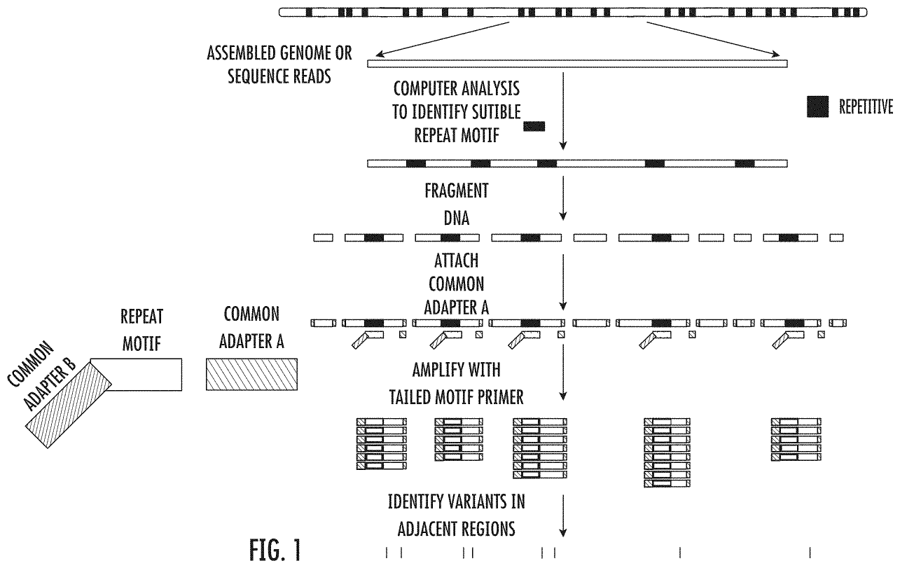

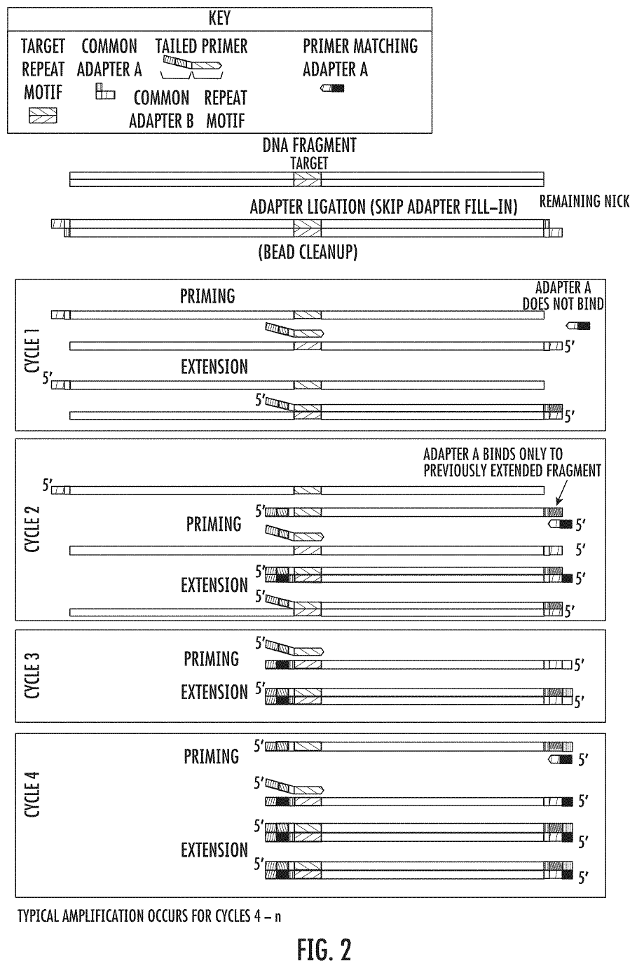

Examples of methods of evaluating genomic variation are now described by referring generally to FIGS. 1 and 2.

A first example of a method for evaluating genomic variation includes (i) generating nucleic acid fragments by fragmenting a nucleic acid, at least one of said nucleic acid fragments comprising a repeat motif; (ii) ligating an adapter molecule having an adapter sequence to the at least one of said nucleic acid fragments including a repeat motif; and (iii) amplifying at least a portion of the nucleic acid fragments using a tailed primer and an adapter primer, said tailed primer including a first nucleic acid sequence complementary to the repeat motif and a second nucleic acid sequence that is at least partially non-complementary to the at least one of said nucleic acid fragments comprising a repeat motif, said adapter primer including a sequence that is homologous to the adapter sequence, thereby producing amplified nucleic acid fragments.

A second example of a method for evaluating genomic variation includes (i) generating nucleic acid fragments by fragmenting a nucleic acid, at least one of said nucleic acid fragments comprising a repeat motif; (ii) ligating an adapter molecule having an adapter sequence to the at least one of said nucleic acid fragments comprising a repeat motif; and (iii) amplifying at least a portion of the nucleic acid fragments using a first tailed primer, a second tailed primer, and an adapter primer, said first tailed primer including a first nucleic acid sequence complementary to the repeat motif and a second nucleic acid sequence that is at least partially non-complementary to the at least one of said nucleic acid fragments comprising a repeat motif, said second tailed primer including a first nucleic acid sequence homologous to the repeat motif, said adapter primer including a sequence at least partially homologous to the adapter sequence, thereby producing amplified nucleic acid fragments.

A third example of a method for evaluating genomic variation includes: (i) identifying a repeat motif in a nucleic acid; (ii) fragmenting the nucleic acid to generate a nucleic acid fragment; (iii) ligating an adaptor molecule having an adapter sequence to an end of the nucleic acid fragment; (iv) annealing a tailed primer to the nucleic acid fragment, the tailed primer having a first nucleic acid sequence that is complementary to the repeat motif and a second nucleic acid sequence that is non-complementary to the nucleic acid fragment; (v) amplifying the nucleic acid fragment via PCR to generate amplified nucleic acid fragments, said PCR using the tailed primer and an adapter primer having a sequence homologous to at least a portion of the adapter sequence; and (vi) sequencing the amplified nucleic acid fragments.

A fourth example of a method for evaluating genomic variation includes: (i) identifying a repeat motif in a nucleic acid; (ii) ligating an adapter to an end of the nucleic acid; and (iii) amplifying the nucleic acid via a PCR protocol using a first tailed primer having a first sequence complementary to the repeat motif and a second sequence non-complementary to the nucleic acid, and a second primer comprising a sequence homologous to the adapter.

A fifth example of a method for evaluating genomic variation includes (i) at least one of identifying and predicting the presence of a repeat motif in a DNA molecule; (ii) fragmenting the DNA molecule to generate DNA fragments; (iii) ligating a first adaptor sequence to at least one of the 5′ and 3′ ends of the DNA fragments; (iv) annealing a primer to the DNA fragments, the primer comprising a first DNA sequence that is complementary to the repeat motif and a second adaptor sequence that is not complementary to the DNA fragment; (v) amplifying the DNA fragments to generate amplified DNA fragments; and (vi) sequencing the amplified DNA fragments to identify regions of genomic variation.

Any of the aforementioned methods may include pooling DNA from diverse species prior to library preparation and simultaneously analyzing the DNA samples. This is possible due to variation in the repeat motifs that exist across the various species. Sequence reads resulting from this pooled approach may be separated out after sequencing using knowledge of which repeat motif exists in each species.

In this context, a sixth example of evaluating genomic variation includes (i) identifying a first repeat motif in a nucleic acid from a first species and identifying a second repeat motif in a nucleic acid from a second species; (ii) pooling the nucleic acids from the first and second species; (iii) generating nucleic acid fragments by fragmenting the nucleic acids; (iv) ligating an adapter molecule having an adapter sequence to at least one of said nucleic acid fragments; and (v) amplifying at least a portion of the nucleic acid fragments using a first tailed primer, a second tailed primer, and an adapter primer, said first tailed primer including a first nucleic acid sequence complementary to the first repeat motif and a second nucleic acid sequence that is at least partially non-complementary to at least one of said nucleic acid fragments, said second tailed primer including a third nucleic acid sequence complementary to the second repeat motif and a fourth nucleic acid sequence that is at least partially non-complementary to at least one of said nucleic acid fragments, said adapter primer including a sequence at least partially homologous to the adapter sequence, thereby producing amplified nucleic acid fragments.

An example of a method for simultaneously evaluating genomic variation in first and second species includes (i) pooling a nucleic acid from the first species having a first repeat motif and a nucleic acid from the second species having a second repeat motif; (ii) generating nucleic acid fragments by fragmenting the nucleic acids; (iii) ligating an adapter molecule having an adapter sequence to at least one of the nucleic acid fragments; and (iv) amplifying at least a portion of the nucleic acid fragments using a first tailed primer, a second tailed primer, and an adapter primer, the first tailed primer including a first nucleic acid sequence complementary to the first repeat motif and a second nucleic acid sequence that is at least partially non-complementary to the at least one of said nucleic acid fragments, the second tailed primer including a third nucleic acid sequence complementary to the second repeat motif and a fourth nucleic acid sequence that is at least partially non-complementary to the at least one of said nucleic acid fragments, the adapter primer including a sequence at least partially homologous to the adapter sequence, thereby producing amplified nucleic acid fragments.

An example of a system for evaluating genomic variation in a nucleic acid fragment having a repeat motif includes a tailed primer including a first nucleic acid sequence complementary to the repeat motif and a second nucleic acid sequence that is at least partially non-complementary to the nucleic acid fragment.

Additional details that may be used in the aforementioned examples of the systems and methods are now described.

The nucleic acid employed in the systems and methods may be any nucleic acid. For example, the nucleic acid may include a DNA molecule defining a sequence of bases adenine (A), guanine (G), thymine (T), and cytosine (C) in any combination. The DNA molecule may include a double-stranded DNA (dsDNA) molecule. Systems and methods using single-stranded DNA (ssDNA) molecules are also envisioned. Under appropriate conditions, RNA may be used.

Nucleic acids including bases other than A, T, G, and C, may also be utilized. For example, a nucleic acid including uracil (U) may be employed. In addition, bases including, but not limited to, synthetic bases may be incorporated into the nucleic acid, such as 3-methyl-6-amino-5-(1′-β-D-2′-deoxyribofuranosyl)-pyrimidin-2-one (S), 6-amino-9[(1′-β-D-2′-deoxyribofuranosyl)-4-hydroxy-5-(hydroxymethyl)-oxolan-2-yl]-1H-purin-2-one (B), 6-amino-3-(1′-β-D-2′-deoxyribofuranosyl)-5-nitro-1H-pyridin-2-one (Z), and 2-amino-8-(1′-β-D-2′-deoxyribofuranosyl)-imidazo-[1,2a]-1,3,5-triazin-[8H]-4-one (P). Any nucleic acid may be used without departing from the teachings presented herein.

The repeat motif(s) used in the systems and methods may be defined by any preferred nucleotide sequence. For example, microsatellites have properties making them very suitable for the systems and methods. Microsatellites are abundant in many genomes, with a number of occurrences that often exceeds 10,000. Moreover, preliminary analyses can be conducted to accurately predict their number, they have been shown to be broadly distributed, and they have also been shown to be generally neutral (i.e. not under selection). Microsatellites are often of sufficient length to allow a suitable melting temperature (see below), and regions immediately downstream from microsatellites are expected to contain diverse, non-repetitive sequences.

Examples of suitable microsatellites include the following underlined short repeating DNA sequences.

[SEQ ID NO: 1]
5′AGTCGTGCTGAATGTGTGTGTGTGTGTGTGACCATCGTAGCTTGC3′
[SEQ ID NO: 2]
5′GCTAGCTCGAGTTGATGATGATGATGATGACTCGGCTAAGATCGA3′

The following is a general representation of an exemplary microsatellite-containing motif: Prefix-Mⁿ-Suffix.

Prefix. The prefix defines the number of bases preceding the microsatellite repeat. This could be zero bases (no prefix), or a nonzero number of bases. Typically, these bases would be degenerate and designed to encourage binding to the beginnings of microsatellite regions. The prefix is useful in allowing the entire microsatellite region to be recovered, thus enabling thousands of microsatellites to be genotyped (i.e. lengths determined) substantially simultaneously.

Mⁿ. Mn denotes a microsatellite with motif M and number of repeats equal to n. For example, TG¹⁰ would signify a TG microsatellite repeated 10 times (total length 20). This motif would have a melting temperature of approximately 60 degrees Celsius.

Suffix. The suffix defines the number of bases following the microsatellite repeat. This could be zero bases (no suffix), or a nonzero number of bases. Typically, these bases would be degenerate and designed to encourage binding to the ends of microsatellite regions. The suffix is useful in producing sequencing containing the maximum amount of usable flanking sequence (for SNP genotyping).

GTⁿ: This motif would bind to any GT microsatellite region longer than n−1 repeats (i.e. to any microsatellite region equal to or longer than n repeats). Tests indicate that if the annealing temperature is lowered, regions with smaller numbers of repeats (down to n−3) can be obtained, even though a motive of length n repeats is used. Although it may bind to multiple places for regions longer than n−1, one would expect that after multiple PCR cycles, the majority of DNA fragments would only contain n repeats, since fragments would only get shorter, not longer. This may be sensitive to the annealing temperature used.

GTⁿ-H, where the degenerate base H means A or C or T (not G): This motif would bind to any GT microsatellite region longer than n−1 repeats, but an extension would only occur if the primer bound to the end of the microsatellite, since most known polymerases do not extend primers that do not match exactly for the last few bases of the template.

GTⁿ-HV, where H means not G and V means not T: Same as the last example, but two degenerate bases are used to increased efficiency.

GTⁿ-A: Same as example GTⁿ-H above, but a non-degenerate base (A) is used to reduce the number of target loci. Preliminary bioinformatic analyses can be conducted to predict the number loci and non-degenerate suffixes or prefixes can be used to fine-tune the number of loci targeted.

V-GTⁿ, where the degenerate base V means not T: In this case the primer will preferentially bind to the beginning of a microsatellite.

HV-GTⁿ, where H means not G and V means not T: Same as the last example, but two degenerate bases are used to increase efficiency.

V-GTⁿ-H, where H means not G and V means not T: In this case the primer would preferentially bind to microsatellites with exactly n repeats.

HV-GTⁿ-HV, where H means not G and V means not T: Same as the last example, but two degenerate bases are used to increase efficiency.

Table 1 depicts the estimated number of non-overlapping occurrences in the human genome of exemplary repeat motifs, based on an analysis of genome build hg38, along with their respective approximate melting temperatures.

TABLE 1

| | | Approx. Melting |
|---|---|---|
| Primer Motif | Expected # In Genome | Temp. (° C.) |
| GT¹⁰ | 51732 | 60 |
| GT¹⁰-H | 51732 | 60-62 |
| GT¹⁰-HV | 16542 | 62-66 |
| V-GT¹⁰ | 29104 | 62-64 |
| HV-GT¹⁰ | 15376 | 64-68 |
| GT¹⁰-A | 30238 | 60 |
| V-GT¹⁰-H | 1798 | 64 |
| HV-GT¹⁰-HV | 234 | 68 |

The exemplary repeat motifs above will recover the flanking regions downstream of the microsatellites (See, e.g., FIGS. 1 and 2). To amplify the regions upstream of a repeat motif, systems and methods employing reverse complements of the motif may be used. In other words, using both TGⁿ and GTⁿ in separate PCR reactions may be used to obtain both sides/flanks of all TG and GT microsatellites, thus doubling the number of genomic regions obtained and thus increasing the segments of a genome analyzed for sequence variation and diversity. This strategy is also useful in improving accuracy when estimating the lengths of microsatellites.

While the examples disclosed herein use the microsatellite GTⁿ as an example, many others are also suitable, e.g., TGⁿ, ACⁿ, CAⁿ, etc., and should be considered to be within the scope of the systems and methods disclosed herein. Moreover, mono-, di-, tri-, tetra-, penta- and hexanucleotide repeats may be used, and some of these longer repeats may be indicative of the presence of a disease or pathogen. Exemplary nucleotide repeats are disclosed in Microsatellites in Different Eukaryotic Genomes: Survey and Analysis, Gábor Tóth et al., Genome Res. 2000 July; 10(7): 967-981, and include, but are not limited to, Aⁿ, Tⁿ, Gⁿ, Cⁿ, ACⁿ, AGⁿ, ATⁿ, CGⁿ, GTⁿ, CTⁿ, AACⁿ, AAGⁿ, AATⁿ, ACCⁿ, ACGⁿ, ACTⁿ, AGCⁿ, AGGⁿ, ATCⁿ, CCGⁿ, GTTⁿ, CTTⁿ, ATTⁿ, GGTⁿ, CGTⁿ, AGTⁿ, GCTⁿ, CCTⁿ, GATⁿ, CGGⁿ, AAATⁿ, AAATⁿ, AGATⁿ, AAAGⁿ, AGATⁿ, ACATⁿ, AAATⁿ, AAATⁿ, AAATⁿ, AAATⁿ, AAAGⁿ, AAAGⁿ, AAAGⁿ, AAGGⁿ, ACAGⁿ, AAATⁿ, ACCTⁿ, AAAGⁿ, ACATⁿ, AAAGⁿ, AAACⁿ, ATCCⁿ, AAACⁿ, AAATⁿ, AAATⁿ, AAACⁿ, AAACⁿ, AAACⁿ, AAGGⁿ, ACTGⁿ, AGCCⁿ, AAAACⁿ, AAAACⁿ, AAAACⁿ, AACTGⁿ, AAAATⁿ, AAAATⁿ, AAAATⁿ, AAAATⁿ, AAAAGⁿ, AAAATⁿ, AAAATⁿ, AAAATⁿ, AGCTCⁿ, AGCTCⁿ, AAAACⁿ, AAAACⁿ, AAATTⁿ, AAAACⁿ, AGATGⁿ, AAAAGⁿ, AAAAGⁿ, AAAATⁿ, CCCCGⁿ, AATATⁿ, AAAACⁿ, AAAAGⁿ, AAAACⁿ, AAAACⁿ, AAAATⁿ, AAAACⁿ, AAGGGⁿ, AAATTⁿ, AAATTⁿ, AAATTⁿ, AGAGGⁿ, AATCGⁿ, AATATⁿ, AGCGGⁿ, AACTGⁿ, ACTATⁿ, AAAAGⁿ, AAACCⁿ, AAACCⁿ, AAATGⁿ, AAACGⁿ, ACTCCⁿ, AACAGⁿ, AATCCⁿ, ATCCCⁿ, ATCCGⁿ, AAAGTⁿ, AAAAGⁿ, AAATCⁿ, AAAAACⁿ, AAAAACⁿ, ACAGGCⁿ, AGAGCGⁿ, AACCCTⁿ, ACAGATⁿ, AAGCCTⁿ, AAAAATⁿ, ACACCCⁿ, AAAAATⁿ, AAAAATⁿ, AAAAATⁿ, AGAGGCⁿ, ACACGCⁿ, AAAAAGⁿ, AACAGCⁿ, AAAAACⁿ, AACAGCⁿ, AACCCTⁿ, AAAAAGⁿ, AAAAAGⁿ, AAAAACⁿ, AAAAACⁿ, AATCCCⁿ, AGCAGGⁿ, AAAAAGⁿ, AAAAAGⁿ, AAAAAGⁿ, ACAGAGⁿ, ACAGCCⁿ, AATAGTⁿ, ACATCCⁿ, AAAAACⁿ, AAAGAGⁿ, AGAGGGⁿ, AGCTCCⁿ, AACTGCⁿ, AAGATGⁿ, AACCAGⁿ, AATGGGⁿ, AAGAGGⁿ, AAATATⁿ, AAAAATⁿ, AATCCCⁿ, AGCTCCⁿ, and AAAAATⁿ.

Adding one or more N's (A, T, C, G or other) to the beginning of the prefix and/or end of the suffix may increase the melting temperature without necessarily reducing the number of target loci recovered.

Any suitable motif may be used without departing from the teachings herein, and the systems and methods disclosed herein are not limited to microsatellite repeats. Once a suitable repeat motif is identified, the repeat motif is employed in connection with the various embodiments of the systems and methods described.

Fragmenting a nucleic acid molecule to generate nucleic acid fragments may include enzymatic digestion of the nucleic acid molecule. For example, fragmenting a DNA molecule with a Fragmentation Through Polymerization (“FTP”) method may produce 300-600 bp dsDNA fragments. An example of a FTP method is disclosed in Fragmentation Through Polymerization (FTP): A new method to fragment DNA for next generation sequencing, Ignatov et al., PLoS One. 2019; 14(4): e0210374.

Generally, FTP includes the steps of (i) nicking a nucleic acid, such as DNA, with a DNAse, such as, for example, DNAse I; and (ii) performing strand displacement with a polymerase, such as, for example, SD polymerase, thereby generating blunt-ended dsDNA fragments with overlapping sequences. One exemplary FTP protocol includes the following:
1. Creating a master mix of:

| | | | |
|---|---|---|---|
| i. | H2O | 10.375 | μL |
| ii. | 10x SD buffer | 2.5 | μL |
| iii. | MgCl2 (100 μM) | 0.875 | μL |
| iv. | dNTP's (25 μM) | 0.25 | μL |
| v. | DNase I (0.1 mg/ml) | 0.25 | μL |
| vi. | SD polymerase | 0.75 | μL |

2. Adding 15 μL master mix to 104 of DNA.
3. Mixing and placing on thermal cycler under the following conditions:
   i. Lid 95° C.
   ii. 30° C. for 20 min
   iii. 70° C. for 20 min
   iv. 10° C. for infinity
4. Adding 25 uL H₂O to sample to bring up to starting volume.
5. Cleaning up the sample using 0.9× SpriSelect beads.
6. Eluting in 20 μL of H2O.

Other nucleic acid fragmentation methods that may be used in the systems and methods will be apparent to one of ordinary skill in the art having the benefit of the present disclosure, and suitable alternatives are considered to be within the scope of this disclosure.

For example, fragmenting a nucleic acid to generate nucleic acid fragments may include sonicating the nucleic acid. Sonicating the nucleic acid may be achieved, for example, with a commercially-available sonication system such as the Covaris® sonication system at 175 Peak Incident Power, 10% duty factor, and 200 cycles per burst for 40 seconds to produce 300-600 bp nucleic acid fragments. Other sonication conditions are possible and may be selected as desired.

Alternatively, enzymatic digestion may be used instead of, or in addition to, sonication to generate the nucleic acid fragments. Other suitable fragmentation techniques may be used without departing from the spirit and scope of the present disclosure.

In some examples, after the fragmenting step, it may be desirable to perform a blunt end repair step. This blunt end repair step may be employed to eliminate any 5′ and/or 3′ overhangs in DNA fragments so that the DNA fragments, or a portion thereof, include double-stranded DNA having blunt ends. If the fragmenting step employs the FTP protocol described above, which itself produces dsDNA fragments with blunt ends, the blunt end repair step may be excluded. In a particular example, a blunt end repair step includes using a solution of T4 polymerase, T4 polynucleotide kinase, dNTPs, and ATP.

The ligating step may include ligating a first adaptor sequence to at least one of the 5′ and 3′ ends of the nucleic acid fragments. The first adaptor sequence may include, for example, a common adapter such as those disclosed in Illumina Sequencing Library Preparation for Highly Multiplexed Target Capture and Sequencing, Meyer and Kircher, Cold Spring Harb Protoc; 2010; doi:10.1101/pdb.prot5448. The first adapter is referred to as Common Adaptor A in FIG. 1. The ligating step may be employed after the aforementioned FTP step or the blunt end repair step, if used. As depicted in FIG. 2, and as described below, the common adapter (first adaptor sequence) provides a sequence from which subsequent amplification steps, such as PCR amplification steps, may be used to selectively amplify the portion of the nucleic acid fragment including the repeat motif.

In some examples of the systems and methods, the ligating step differs from a standard library preparation protocol because only P7 adapters are ligated. Common Adapter A (first adaptor sequence) may therefore include a P7 adapter. In standard Library preparation (Meyer and Kircher, 2010), P5 and P7 adapters are ligated to the ends of DNA fragments after blunt end repair. In some examples of the systems and methods, P7 adapters are ligated as a control measure. In certain examples, the “tailed primer” has the P5 adapter as its tail (i.e. the sequence that is non-complementary to the nucleic acid fragment, for example). This ensures that only nucleic acid fragments that have been amplified with the “tailed primers” are able to be sequenced.

The systems and methods may include an amplifying step. In certain examples, the amplifying step includes a polymerase chain reaction or “PCR” protocol. Although the exemplary PCR protocols disclosed herein employ the Phusion® Polymerase, other polymerases, including commercially-available thermostable polymerases, may be used without departing from the teachings of the present disclosure. Likewise, tailed primer concentrations may be modified in order to optimize PCR output and on-target percentages, and annealing temperatures may be modified to optimize PCR output and sequencing on-target percentage.

The amplifying step may include annealing a tailed primer to the nucleic acid fragments as part of a PCR amplification protocol, wherein the tailed primer includes a first nucleic acid sequence that is complementary to the repeat motif and a second adaptor sequence that is not complementary to the nucleic acid fragment. In one or more embodiments, the annealing temperature comprises between about 58-80 degrees Celsius.

The second adapter sequence (Common Adapter B in FIG. 2) may include a P5 adapter upstream of, or followed by, a DNA sequence that is complementary to the repeat motif (when considered in the 5′ to 3′ direction). As depicted in FIGS. 1 and 2, this results in the primer only partially annealing to the DNA fragment. Exemplary second adapter sequences such as a P5 adapter are useful in downstream processing, such as DNA sequencing. At least one of the first and second adapter sequences may also include a “barcode” sequence for subsequent identification of a desired fragment or sample by its sequence. The barcodes may be the same, or they may be different.

As depicted in FIG. 2, exemplary systems and methods of the present disclosure provide for the selective amplification of DNA fragments having the identified repeat motif. Once Common Adaptor A (first adaptor sequence), for example a P7 adapter, is ligated to the ends of the nucleic acid fragment, a PCR protocol is initiated. By way of non-limiting example, cycle 1 of the PCR protocol includes priming with a tailed primer having a 5′ Common Adapter B (second adapter sequence) such as, for example a P5 adapter, and a 3′ repeat motif. The 3′ repeat motif, in this instance, is complementary to the target repeat motif of the DNA fragment. In cycle 1, a primer containing the Common Adapter A sequence, which includes an additional 5′ region, does not bind, and only one strand of the DNA fragment is amplified by elongation of the tailed primer by the polymerase.

In cycle 2, the tailed primer provides a point from which a polymerase elongates the strand of the DNA fragment having the target repeat motif, and the primer matching Common Adaptor A binds only the previously elongated fragment. In cycles 3 and 4, the fragment having the repeat motif is amplified using both the tailed primer and the primer matching Common Adaptor A as depicted in FIG. 2.

The amplified nucleic acid fragments may be subjected to quality control with at least one of Qubit and Bioanalyzer. Moreover, the amplified DNA fragments may be selected by size using Pippin HT or some other DNA size selection method.

Some of the systems and methods may include sequencing the amplified nucleic acid fragments using conventional nucleic acid sequencing techniques.

In certain examples, the first nucleic acid sequence that is complementary to the repeat motif is preceded by (i.e. upstream of or 5′ to) the second adaptor sequence that is not complementary to the nucleic acid fragment. Other suitable configurations, however, are within the scope of the disclosure. The nucleic acid molecule may be from a particular species of interest.

If degenerate bases in the primer are used to anneal the primer to the beginning of the repeat motif, the length of the repeat motif may itself be assessed and considered genomic variation of interest. Thus, the systems and methods may also be used to evaluate the lengths of the repeat motifs themselves (i.e. measuring microsatellite lengths for a motif across the genome, for example), since the lengths of these motifs may be the fastest type of genomic variation.

In order to evaluate regions of a genome both upstream and downstream from a repeat motif, a second tailed primer including a first nucleic acid homologous to the repeat motif and a third nucleic acid sequence that is at least partially non-complementary to the nucleic acid fragment may be used. The second and third nucleic acid sequences may homologous or non-homologous, depending on the application.

In applications seeking to simultaneously evaluate genomic variation across divergent species, the nucleic acid fragment may be multiple nucleic acid fragments from divergent species.

In the systems and methods, the identifying step may include employing a bioinformatics protocol that identifies and/or selects the repeat motifs. The bioinformatics protocol may be performed by and/or with the aid of a software application configured for performing the various steps of the protocol.

An example of a bioinformatics protocol includes (a) loading genome sequences in a format, such as, for example, a fasta (assembled) or fastq (raw reads) format; and (b) using HashMap or similar data structure to store a sample of Kmers (short DNA sequences) with melting temperature (Tm), with key=Kmer and value=count. Selection of a particular melting temperature in (b) ensures that repeat motifs will be viable for the PCR reaction.

The bioinformatics protocol may also include the following features:
1. Starting at each position in the genome file, increasing K until the Kmer reaches the desired Tm;
2. If the Kmer is already in the hash table, increment counter;
3. If not, add Kmer to hash table and set initial count to 1; and
4. After some percentage of the genome (e.g. 10%) has been evaluated, only increase count (do not add additional Kmers). This reduces RAM requirements.

The bioinformatics protocol may also include (c) profiling each Kmer for genomic abundance to identify a candidate. This step (c) ensures that the desired number of loci will be recovered with PCR.

The bioinformatics protocol may include:
1. Using HashMap counts to predict the total number of times each Kmer exists in genome; and
2. Disqualifying Kmers with below some threshold (e.g. 1000). The remainder are candidates.

The bioinformatics protocol may also include (d) profiling each candidate for potential to mis-prime. This step (d) ensures that a high proportion of recovered reads will map to the target regions.

The bioinformatics protocol may include:
1. Using a second HashMap to identify abundance of each Kmer that is one base pair different than one or more of the candidates (one-offs); and
2. Disqualifying candidates with too many one-offs in genome (high potential to mis-prime).

The bioinformatics protocol may also include (e) profiling each candidate for sequence diversity in downstream flank (e.g. 100 bp). This step (e) ensures that assembly of recovered reads will be possible.

The bioinformatics protocol may include:
1. Hashing all short Kmers (e.g. K=5) observed in flanks, noting flank position and which candidate to which the flank corresponds. At the end of this process a binary table exists for each candidate indicating whether each of the possible 4^{K} possible Kmers exists at least once at some distance from the Kmer;
2. Using the binary tables to compute the number of unique Kmers present at each position in the flank of each Kmer. This is a measure of sequence diversity;
3. For each candidate, identifying the flank position at which the diversity is not elevated; and
4. Disqualifying candidates for which the diversity does not return suitable levels within some distance of the Kmer (e.g. 50 bp). This ensures that chosen repeat motifs are positioned on edge of repetitive region, if desired. Alternatively, one or more embodiments may comprise allowing candidates having high diversity in the flanking region, which may be beneficial for various applications of the systems and methods disclosed herein.

The bioinformatics protocol may also include (f) profiling each candidate for genomic uniformity. This step (f) ensures that repeat motifs will not be selected from tandemly repeated genomic regions.

The bioinformatics protocol may include:
1. Counting the proportion of times each Kmer occurs in some proximity (e.g. 1000 bp) to itself in the genome; and
2. Disqualifying candidates for which this proportion is too high (e.g. 10%).

The bioinformatics protocol may include (g) profiling each candidate for levels of selection (if desired). This step ensures that repeat motifs will not be selected from regions under selection.

The bioinformatics protocol may include:
1. Using existing profiles of genome wide strengths of selection to identify candidates with flanks that are under selection. If profiles do not exist, using heterozygosity profiles from the samples of interest instead; and
2. Disqualifying candidates for which selection is identified to be too strong or too weak.

The bioinformatics protocol may also include (h) collapsing similar Kmers using degenerate bases (if desired). This step allows repeat motifs to encompass several related sequences and may reduce allelic dropout, etc. The bioinformatics protocol may include:
1. Clustering Kmers by similarity; and
2. Creating degenerate motifs for each cluster.

The bioinformatics protocol may also include (i) evaluating alignments of flanking regions of candidates.

The bioinformatics protocol may include:
1. Selecting several candidates with suitable profiles;
2. Extracting flanking regions for some sample of the candidate's occurrences;
3. Viewing these sequences as an alignment; and
4. Verifying that the regions are suitable (e.g. library diversity is high).

The bioinformatics protocol may also include (j) evaluating the potential for each candidate to be a suitable primer.

The bioinformatics protocol may include:
1. Determining whether the primer will self-anneal;
2. Determining whether the primer will anneal to one of the common adapter sequences; and
3. Disqualifying if a or b is true.

The bioinformatics protocol may include (k) selecting at least one suitable repeat motif for use in subsequent steps in the method. For example, as described below, the methods may include appending the suitable repeat motif sequence to a common adapter sequence for use in connection with the various examples of the systems and methods.

### EXAMPLES

To illustrate the effectiveness of certain examples disclosed herein and their advantages, the bioinformatics protocol was used to perform a computer analysis of published genome sequences and identify at least two repetitive elements for each of six model species and designed the corresponding lab reagents. Lab reagents were applied to DNA from the same six species in order to subsample the DNA prior to sequencing. The post-sequencing analysis indicates that for all six species, 95% of the expected genomic regions with <10% of the DNA sequences wasted (not mapped to an expected region) were obtained.

Primer Development. The Six model species in Table 2 were selected based on taxonomic diversity, genome size, and genome assembly quality.

TABLE 2
Model Species

| | Genome | Genome Size | Genome |
|---|---|---|---|
| Species | Build | (Gb) | Quality |
| Human | hg38 | 3.5 | Excellent |
| Clawed Frog | xenTro9 | 1.7 | Good |
| Zebrafish | danRer11 | 1.5 | Good |
| Silkworm | bomMor1 | 0.5 | Fair |
| Corn | B73_RefGen_v4 | 2.3 | Fair |
| Soybean | Glycine_max_v2.1 | 1.1 | Good |

Unmasked versions of the assembled genomes were downloaded, then profiled for suitable repeat motifs using the bioinformatics protocol. Two repeat motifs were selected for each of the six species. For each motif, we designed a tailed primer containing the common P5 adapter (with 8 bp unique barcode indexes) followed by the repeat motif. The tailed primer was synthesized by IDT and purified using PAGE purification process to increase the proportion of full-length primers. The tailed primer sequences were as follows, with barcode indexes in bold and repeat motif underlined:

Human 1
[SEQ ID NO: 3]
5′AATGATACGGCGACCACCGAGATCTACACA
ATAGCAAACACTCTTTCCCTACACGACGCTCT
TCCGATCTGAGATTTGGGTGGGGACACA3′
Human 2
[SEQ ID NO: 4]
5′AATGATACGGCGACCACCGAGATCTACACA
CTCGCTAACACTCTTTCCCTACACGACGCTCT
TCCGATCTTGGTGCCAAAAAGGTTGGGG3′
Frog 1
[SEQ ID NO: 5]
5′AATGATACGGCGACCACCGAGATCTACACA
GGCCTTGACACTCTTTCCCTACACGACGCTCT
TCCGATCTCCACTGGTTGGGGATCACTG3′
Frog 2
[SEQ ID NO: 6]
5′AATGATACGGCGACCACCGAGATCTACACA
TTGAAGGACACTCTTTCCCTACACGACGCTCT
TCCGATCTTTGGCAGTAAAATGCCAAAA3′
Fish 1
[SEQ ID NO: 7]
5′AATGATACGGCGACCACCGAGATCTACACC
CAAGAGTACACTCTTTCCCTACACGACGCTCT
TCCGATCTGGTGTGAAAACACCCTGCTG3′
Fish 2
[SEQ ID NO: 8]
5′AATGATACGGCGACCACCGAGATCTACACC
GATGACGACACTCTTTCCCTACACGACGCTCT
TCCGATCTGGGGGTTTCATGGCCCTTTA3′
Silkworm 1
[SEQ ID NO: 9]
5′AATGATACGGCGACCACCGAGATCTACACC
TCGAACCACACTCTTTCCCTACACGACGCTCT
TCCGATCTATTTTAAATGCCCAGCGAAG3′
Silkworm 2
[SEQ ID NO: 10]
5′AATGATACGGCGACCACCGAGATCTACAC
GAGCATACACACTCTTTCCCTACACGACGCT
CTTCCGATCTCGCGTTCAAACAAACAAACT3′
Corn 1
[SEQ ID NO: 11]
5′AATGATACGGCGACCACCGAGATCTACACT
CCTGAGAACACTCTTTCCCTACACGACGCTCT
TCCGATCTATTCACCCCCTCTAGGCGAC3′
Corn 2
[SEQ ID NO: 12]
5′AATGATACGGCGACCACCGAGATCTACACT
TCAGCAGACACTCTTTCCCTACACGACGCTCT
TCCGATCTACGCACGGGCACTCACCTAG3′
Soybean 1
[SEQ ID NO: 13]
5′AATGATACGGCGACCACCGAGATCTACACG
CTCTGCTACACTCTTTCCCTACACGACGCTCT
TCCGATCTAATTCAACCCCCCCTTCTTA3′
Soybean 2
[SEQ ID NO: 14]
5′AATGATACGGCGACCACCGAGATCTACACG
TCGCTAGACACTCTTTCCCTACACGACGCTCT
TCCGATCTTTCAYCATGAAGCTTTGCTT3′

DNA Fragmentation. Respective DNA molecules were fragmented by sonication using the commercially-available Covaris® sonication system at 175 Peak Incident Power, 10% duty factor, and 200 cycles per burst for 40 seconds to produce 300-600 bp nucleic acid fragments.

Blunt End Repair. After sonication, a blunt end repair step was performed via the following protocol:
1. Create a master mix, the master mix comprising:

| | | | | |
|---|---|---|---|---|
| i. | H20 | 7.12 | μL | |
| ii. | Buffer Tango | 7 | μL | Thermo Fisher Cat. # BΥ5 |
| iii. | dNTP's (25 mM) | 0.28 | μL | |
| iv. | ATP (100 mM) | 0.7 | μL | |
| v. | T4 PNK | 3.5 | μL | Epicentre Cat. # P0503K |
| vi. | T4 Polymerase | 1.4 | μL; | New England Biolabs Cat. #M0203L |

2. Add 20 μL of the master mix to 50 μL of a sonicated sample.
3. Mixing and placing on thermalcycler using the following conditions:
   i. Lid OFF
   ii. 27° C. for 15 min
   iii. 12° C. for 5 min
   iv. 4° C. hold
4. Clean up the sample using 0.9× SpriSelect beads.
5. Elute in 20 μL of H20.

Adapter Ligation. After blunt end repair, adapters were ligated to the DNA fragments using the following protocol:
1. Creating a P7 mix, the mix comprising:

| | | | |
|---|---|---|---|
| i. | IS2_adapter P7.f (500 μM) | 20 | μL |
| ii. | IS3_adapter P5 + 7.R (500 μM) | 20 | μL |
| iii. | Oligo Hybridization Buffer (10x) | 5 | μL |
| iv. | H20 | 10 | μL; |

2. Creating a master mix, the master mix comprising:

| | | | | |
|---|---|---|---|---|
| i. | H20 | 10 | μL | |
| ii. | T4 Ligase Buffer | 4 | μL | Thermo Scientific Cat # EL0012 |
| iii. | PEG-4000 | 4 | μL | Thermo Scientific Cat # EL0012 |
| iv. | P7 Mix | 1 | μL | |
| v. | T4 Ligase | 1 | μL; | Thermo Scientific Cat # EL0012 |

3. Add 20 μL of master mix to 20 μL of blunt end repair product.
4. Mixing and placing on thermalcycler using the following conditions:
   i. 22° C. for 30 min
   ii. 4° C. hold.
5. Clean up using 1.8× AmpureXP beads.
6. Elute in 20 μL of H20.

Amplification. Exemplary one-step and two-step PCR reactions are disclosed below. For the data presented herein, amplifying steps were performed using the one-step PCR protocol.

### A) Two-Step Polymerase Chain Reaction

a. Create master mix

| | | | |
|---|---|---|---|
| i. | H20 | 17.1 | μL |
| ii. | Phusion Buffer (10x) | 10 | μL |
| iii. | dNTP's (25 mM) | 0.4 | μL |
| iv. | Tailed Repeat Motif Primer (10 μM) | 1 | μL |

| | | |
|---|---|---|
| v. | Phusion Polymerase | 0.5 μL (New England |
| | | Biolabs Cat. # M0535L) |

b. Add the following to each sample well:

| | | | |
|---|---|---|---|
| i. | 17 index (10 μm) | 1 | μL |
| ii. | Ligation product | 20 | μL |
| iii. | Master mix | 29 | μL |

c. Mix and place on thermalcycler for program “MP_IND_5 cyc3”
   i. Lid: 100° C.
   ii. 98° C. for 45 sec
   iii. 98° C. for 10 sec
   iv. 58° C. for 20 sec
   v. Cycle to (ii) 5 times
   vi. 72° C. for 10 min
   vii. 4° C. for infinity
d. Cleanup with 1.8× AmpureXP beads
e. Elute in 25 μL of H₂O
f. Create master mix

| | | | |
|---|---|---|---|
| i. | H2O | 15.55 | μL |
| ii. | Phusion Buffer (10x) | 5 | μL |
| iii. | dNTP's (25 mM) | 0.2 | μL |
| iv. | P5 outer adapter (10 μM) | 0.5 | μL |
| v. | P7 outer adapter (10 μM) | 0.5 | μL |

| | | |
|---|---|---|
| vi. | Phusion Polymerase | 0.25 μL (New England Biolabs |
| | | Cat. # M0535L) |

g. Add the following to each sample well:

| | | | |
|---|---|---|---|
| i. | Master mix | 22 | μL |
| ii. | PCR 1 product | 3 | μL |

h. Mix and place on thermalcycler for program “IS5_6_68”
   i. Lid: 100° C.
   ii. 98° C. for 45 sec
   iii. 98° C. for 12 sec
   iv. 68° C. for 10 sec
   v. Cycle to step (ii) 12 times
   vi. 72° C. for 10 min
   vii. 4° C. for infinity
i. Cleanup with 1.8× AmpureXp beads
j. Elute in 35 uL H2O and proceed to step 5

B) One-Step Polymerase Chain Reaction
a. Create master mix

| | | | |
|---|---|---|---|
| i. | H20 | 17.35 | μL |
| ii. | Phusion Buffer (10x) | 10 | μL |
| iii. | dNTP's (25 mM) | 0.4 | μL |
| iv. | P5 outer adapter (10 μM) | 0.25 | μL |
| v. | P7 outer adapter (10 μM) | 0.25 | μL |
| vi. | Tailed Repeat Motif Primer (10 μM) | 0.25 | μL |

| | | |
|---|---|---|
| vii. | Phusion Polymerase | 0.5 μL (New England |
| | | Biolabs Cat. # M0535L) |

b. Add the following to each sample well:

| | | | |
|---|---|---|---|
| i. | 17 index (2.5 μM) | 1 | μL |
| ii. | Ligation product | 20 | μL |
| iii. | Master mix | 29 | μL |

c. Mix and place on thermalcycler for program “IS5_6_25”
   i. Lid: 100° C.
   ii. 98° C. for 45 sec
   iii. 98° C. for 12 sec
   iv. Primer melting temperature for 30 sec
   v. 72° C. for 20 sec
   vi. Cycle to (ii) 25 times
   vii. 72° C. for 10 min
   viii. 4° C. for infinity
d. Cleanup with 1.8× AmpureXP beads
e. Elute in 35 μL of H₂O

The method used for cleaning up the DNA samples was as follows:
1) Calculating amount of beads needed (0.9× for SpriSelect, 1.8× for AmpureXP)

| | REPAIR | LIG8_22 | Post-PCR | IS5&6_68 |
|---|---|---|---|---|
| beads | SpriSelect | AmpureXP | AmpureXP | AmpureXP |
| ratio | 0.9 | 1.8 | 1.8 | 1.8 |
| Vol input | 70 | 40 | 50 | 25 |
| Vol | 63 | 72 | 90 | 45 |
| needed | | | | |

2) Add bead volume to each sample well.
3) Pipette 7-10× to thoroughly mix.
4) Incubate at room temperature for 5 min to allow DNA to bind to the beads.
5) Place plate on magnet for 5 min to allow beads to separate from supernatant.
6) Remove exact volume of supernatant (do NOT remove any beads).
7) Wash beads with 150 μL of freshly made 70% ethanol.
8) Remove all ethanol.
9) Repeat wash with 150 μL of ethanol.
10) Removing all Ethanol.
11) Dry samples at room temperature for 5-10 minutes or until the bead ring is no longer shiny, and appears cracked.
12) Add elution volume+5 μL of H₂O to the dried bead rings.
13) Mix 7-10× to thoroughly mix the sample.
14) Incubate at room temperature for 5 min to allow DNA to separate from beads.
15) Place plate on magnet for 5 min to allow beads to separate from supernatant.
16) Transfer supernatant to a clean tube/plate.

Sample Analysis and Sequencing. DNA from each species was enriched for each repeat motif using the methods described above. Four replicates were performed using the one-step protocol with four different annealing temperatures (58.0, 60.5, 67.7, 70.0). DNA concentrations were assessed using Qubit™ Fluorometric Quantification (ThermoFisher Scientific), library size distributions were evaluated using a Bioanalyzer (Agilent), and library quality/quantity was determined using Kapa qPCR (Kapa Biosystems, Inc.). Libraries were pooled in equal volumes and sequenced on an Illumina NovaSeq 6000 sequencer, with a paired-end 150 bp protocol. A total of 41 Gb of raw sequence reads were collected, corresponding to a predicted sequencing effort of 200-fold coverage per target locus.

Bioinformatics Analysis. Reads were demultiplexed with no mismatches tolerated. This resulted in one pair of read files per motif (12 pairs per annealing temperature). Overlapping paired reads were merged and adapters were removed. Unmerged read pairs were not analyzed downstream. We trimmed the first 50 bp from each merged read, as this region contained the repeat motif and some additional low-complexity sites. The trimmed reads were then mapped to the genome from which the motifs were derived.

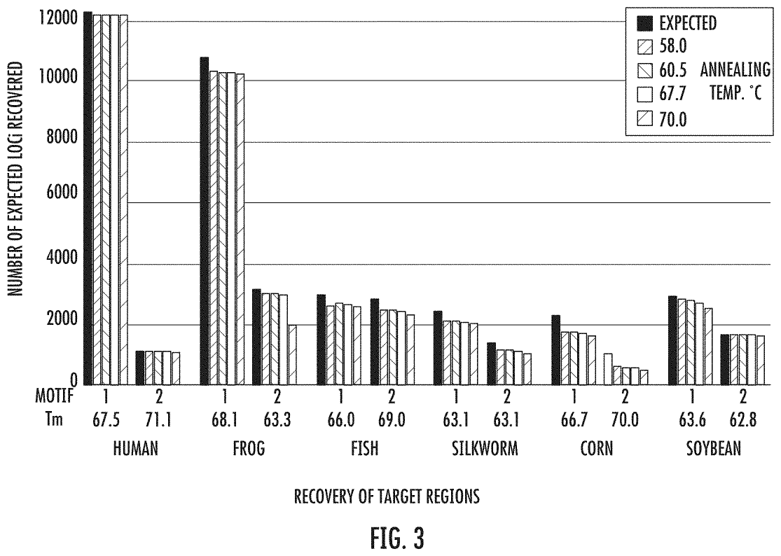

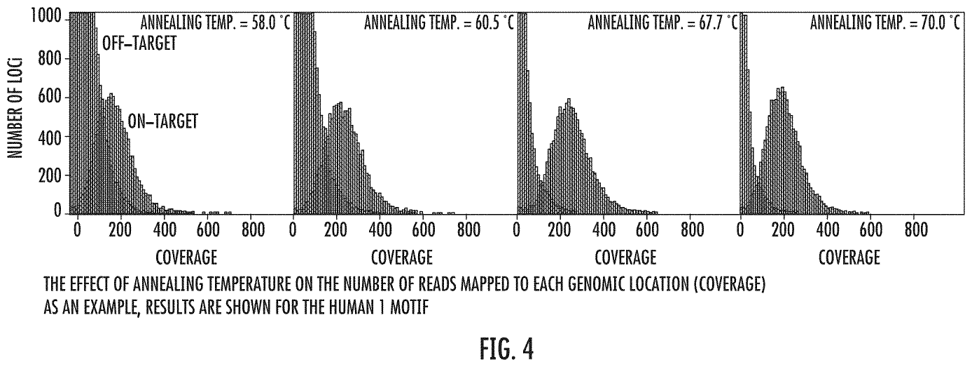

Results. As depicted in FIGS. 3 and 4, both primer motifs in each of the six species were able to recover a large portion of the target loci. Raising annealing temperature increased the proportion of reads that mapped to target regions but decreased the number of target loci recovered to a small degree. In general, performance was best for species with the highest-quality genomes.

To further demonstrate the utility of the systems and methods, a primer containing a GT11 repeat was used to amplify >75,000 loci in a human sample (T_{A}=78.5° C., Primer Conc.=0.25 μM). The results showed very high efficiency (low off-target mapping), as well as the characteristics of the enriched region, which contains a short repetitive region (30 nucleotides matching primer) and a longer non-repetitive region (˜200 sites containing sequence to be used downstream).

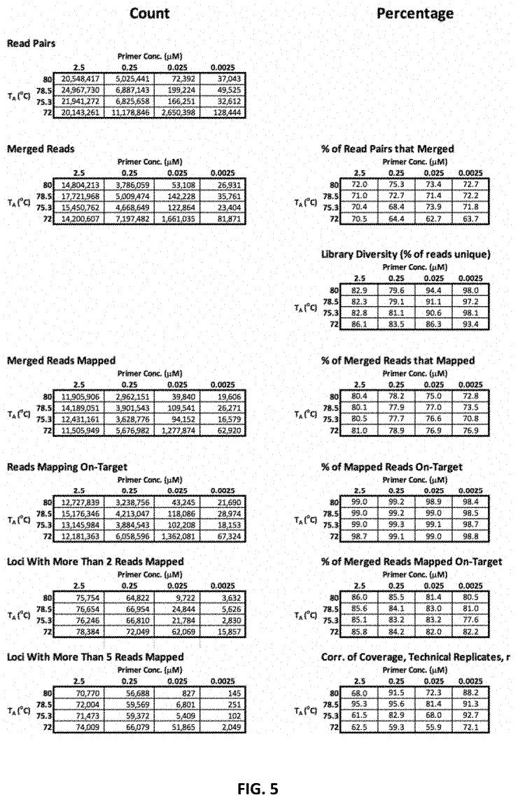

FIG. 5 reveals the effect of annealing temperature (T_{A}, ° C.) and primer concentration (μM) on efficiency when a GT11-containing primer is used to enrich human DNA. Libraries were constructed from human DNA as described in herein, pooled in equal molar concentrations, then sequenced on an Illumina NovaSeq 6000 sequencer with a paired-end 150 bp protocol. After demultiplexing using two barcodes (to sort reads by PCR condition), overlapping read pairs were merged then mapped to the hg38 build of the human genome. Regions within 100 bases of a GT microsatellite (length>8 repeats) were considered on-target. Results demonstrate that loci can be efficiently obtained under a variety of PCR conditions.

The headings presented in FIG. 5 are defined as follows.

Read pair. The Illumina sequencer can be configured to produce two reads from each library insert, each beginning at one of the ends and extending towards the middle of the insert. Inserts of length less than two times the read length will produce reads that overlap in the middle. The two reads can be lined up and merged into a single read. In the following example, a fragment of length 50 nt is sequenced with a paired-end 30 nt protocol. The two resulting reads overlap by 10 nt in the middle, producing a 50 nt merged read.

Library Insert:
[SEQ ID NO: 15]
5′ ACAGACATTTACAGTATACGGATGACTAGC
ATTTAGCTTAGCTATCCTAC 3′
Read 1:
[SEQ ID NO: 16]
5′ ACAGACATTTACAGTATACGGATGACTAGC 3′
Read 2:
[SEQ ID NO: 17]
3′ CTACTGATCGTAAATCGAATCGATAGGATG 5′
Merged Read:
[SEQ ID NO: 18]
5′ ACAGACATTTACAGTATACGGATGACTAGCATT
TAGCTTAGCTATCCTAC 3′

Merged read. The sequence read of an entire library insert, reconstructed by combining an overlapping read pair. Read pairs that cannot be merged typically come from library inserts that are longer than two times the read length.

Library Diversity. The percentage of sequenced reads that are derived from different fragments of original sample DNA. Low diversity indicates that many of the sequence reads are derived from PCR copies of the same DNA fragment.

Merge Reads Mapped. The number/percentage of merged reads that could be mapped to (placed on) the human genome reference.

Reads Mapping On-Target. The number/percentage of reads whose mapping position was within 100 nt of human genome position that contained a GT microsatellite, as determined by an analysis of the reference human genome.

Corr. Of Coverage, Technical Replicates. r. The experiment was conducted twice independently under each of the 16 conditions. After merging overlapping reads and mapping the merged reads to the human genome reference, the read coverage (sequencing depth) at corresponding loci were compared between the two technical replicates. The Correlation Coefficient, r, was computed to represent the correspondence in read coverage between the two technical replicates.

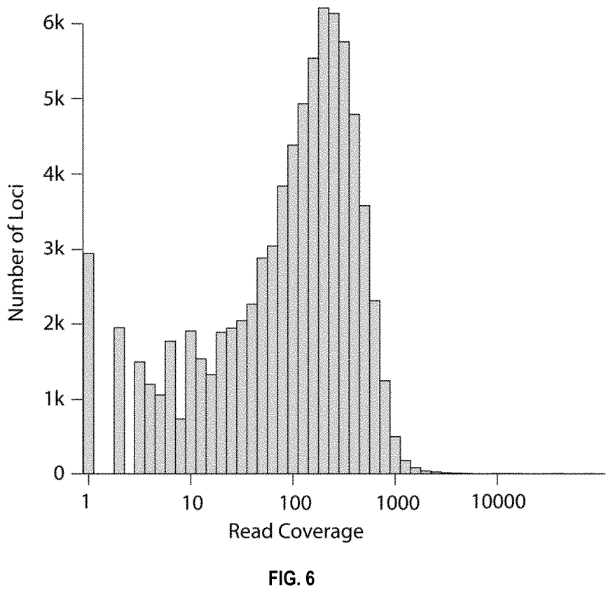

FIG. 6 depicts the distribution of read coverage across loci. Using a GT11-containing primer, >70,000 loci were isolated from human DNA (T_{A}=78.5° C., Primer Conc.=0.25 μM). More than 100 reads were obtained for a large majority of the target loci.

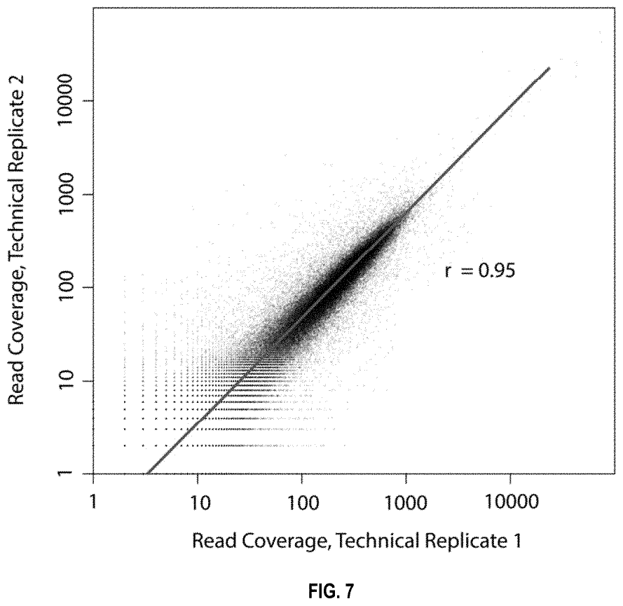

FIG. 7 depicts a correlation of coverage between technical replicates. A GT11-containing primer was used to enrich human DNA for >75,000 loci (T_{A}=78.5° C., Primer Conc.=0.25 μM) in two technical replicates (the laboratory protocol was repeated twice independently). Each point in the graph represents one locus. The correlation between the technical replicates is very high (r=0.95) indicating that the method is useful for evaluating genetic variation at the same set of loci for multiple samples.

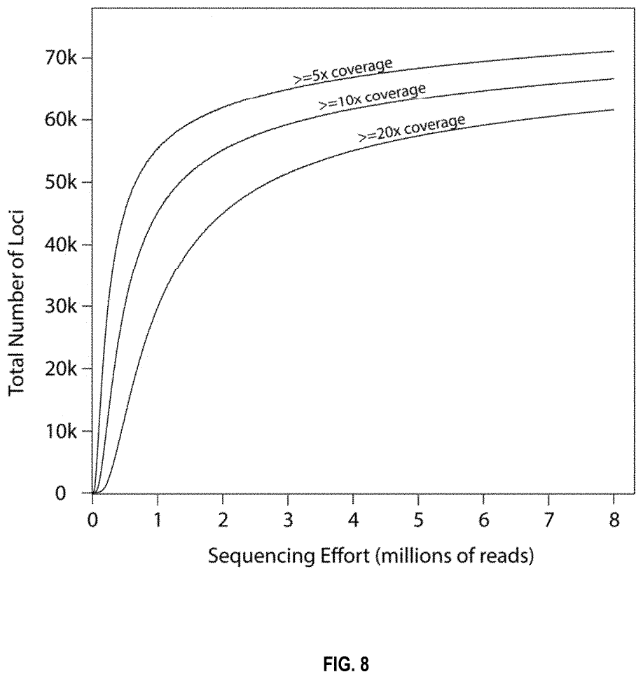

FIG. 8 depicts the relationship between sequencing effort and the total number of loci obtained when a GT11-containing primer is used to enrich human DNA (T_{A}=78.5° C., Primer Conc.=0.25 μM). The relationship is shown given three coverage requirements for counting a locus. Note that a large number of loci (e.g. >30 k) can be obtained with a small sequencing effort (e.g. 1 million reads). The actual cost of sequencing each sample can be estimated by converting the X-axis into dollars (assuming the current per-read sequencing cost) and choosing a target number of loci and coverage threshold.

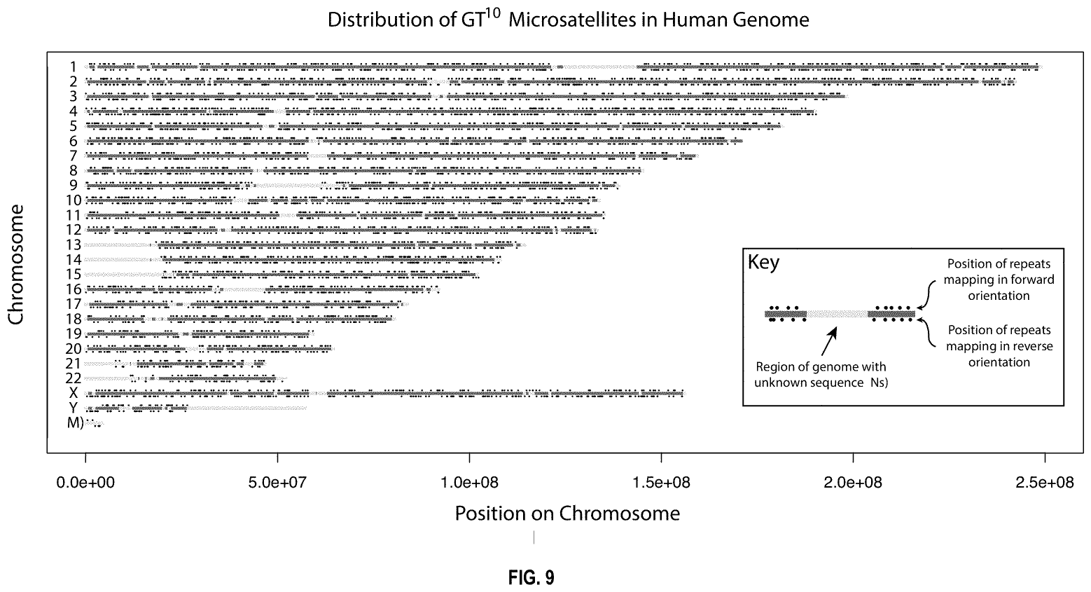

The disclosed bioinformatics protocol was used to identify an exemplary repeat motif for use in conjunction with the disclosed systems and methods. FIG. 9 depicts the distribution of GT¹⁰ microsatellites in human genome build hg38. The results presented in FIG. 9 reveal the utility and advantages of the disclosed systems and methods for identifying and employing repeat motifs that are widely distributed throughout a genome, thereby enabling an evaluation of genomic variation across many loci.

FIGS. 5-8 highlight further advantages of the disclosed systems and methods over preexisting systems.

Cost. The cost to collect data with this example of the method is lower than that of preexisting systems, such as ddRAD. Data are currently collected for about $5 per sample ($2 for library preparation and $3 for sequencing, depending on desired coverage—FIG. 8). For competitive systems and methods, the cost per sample is about $68 per sample ($35 library preparation, $23 sequencing, an $10 for administration etc.).

Data Quantity. The method allows for customizing data sets to a customer's needs, from a few thousand loci to hundreds of thousands of loci. The method is more efficient, especially when large numbers of loci are desired (See FIG. 5). The cost estimate given above is based on use of the method to collect about 75,000 loci per individual. For most competitive systems, data output is only about 5,000-10,000 loci at higher cost. Therefore, the method can be used to collect more data at a lower cost.

Data Quality. A drawback of preexisting systems is relatively low repeatability. Due to the stochastic nature of the DNA fragment size selection process that is critical to ddRAD, for example, levels of read coverage across loci can be quite variable from sample to sample. This leads to missing data when samples are compared downstream. The method discussed here, in contrast, has high repeatability across samples, as seen in FIG. 7. Moreover, as shown in FIG. 6, the read coverage across loci is relatively consistent.

Another drawback of preexisting systems, like ddRAD, is that the composition of the data (the exact set of loci recovered) can be sensitive to point mutations. This is because it relies on restriction enzymes that digest the genome at specific, short motifs. One point mutation at a restriction site will cause the enzyme to not cut the DNA at that site, thus affecting the size of fragments in that region of the genome. These fragment size shifts can change the distribution of loci that are size selected. Due to this limitation (as well as the highly stochastic nature of size selecting DNA fragments mentioned above), it is common to have low correspondence across samples with respect to loci that are obtained. The resulting data matrix is often patchy with portions of missing data. The methods and systems disclosed herein overcome these limitations.

Genomic Representation. The systems and methods disclosed herein and preexisting systems can both be used to sample loci that are broadly distributed across the genome, although base composition (GC content) could affect the uniformity of these distributions to some degree.

Temporal Scale of Applicability. Preexisting systems are typically used to collect SNP data for within-species studies. Across species applications are less common because the reproducibility of the method declines sharply as the evolutionary time among samples increases: when samples are taken from across species separated by millions of years, the number of sampled loci that the samples have in common tends to be very low.

The systems and methods disclosed herein are useful for comparing samples taken from within or across species. The reason for this is the relatively long lifespan of repetitive elements. Although microsatellites and other repetitive elements can be rapidly evolving (i.e. by changing length), the results of the systems and methods are robust to these changes (changes in length has a relatively small effect on priming efficiency). This robustness results in large overlap in the sets of loci obtained by related species.

Lastly, since microsatellites lengths evolve at a rate 3-5 times faster than non-repetitive areas (i.e. restriction sites), the ability to ascertain the lengths of tens of thousands of microsatellites (in addition to obtaining SNP data in the flanking region) allows the systems and methods to be used at the shallowest of scales, including important applications in humans (i.e. for forensics, paternity testing, and ancestry, etc.).

The systems and methods are not limited to the details described in connection with the example embodiments. There are numerous variations and modification of the systems and methods that may be made without departing from the scope of what is claimed.

What is claimed is:

1. A method for evaluating genomic variation, the method comprising:
generating nucleic acid fragments by fragmenting a nucleic acid, at least one of said nucleic acid fragments having a repeat motif;
ligating an adapter molecule having an adapter sequence to the at least one of said nucleic acid fragments having a repeat motif; and
amplifying at least a portion of the at least one of said nucleic acid fragments having a repeat motif using a tailed primer and an adapter primer, said tailed primer including a first nucleic acid sequence that binds to the repeat motif and a second nucleic acid sequence that does not bind to the at least one of said nucleic acid fragments having a repeat motif, said adapter primer including a nucleic acid sequence homologous to the adapter sequence, thereby producing amplified nucleic acid fragments.

2. The method of claim 1, wherein the repeat motif includes a nucleotide sequence including at least one of GTⁿ, GTⁿ-H, GTⁿ-HV, GTⁿ-A, V-GTⁿ, HV-GTⁿ, V-GTⁿ-H, HV-GTⁿ-HV, TGⁿ, ACⁿ, CAⁿ, and a reverse complement thereof.

3. The method of claim 1, wherein the first nucleic acid sequence is complementary to the repeat motif.

4. The method of claim 1, wherein the second nucleic acid sequence is at least partially non-complementary to the at least one of said nucleic acid fragments having a repeat motif.

5. The method of claim 1, wherein the fragmenting comprises sonicating the nucleic acid.

6. The method of claim 1, wherein the first nucleic acid sequence is downstream of the second nucleic acid sequence.

7. The method of claim 1, further comprising selecting the repeat motif using a bioinformatics protocol comprising:
(a) loading a nucleic acid sequence into a software program;
(b) using a data structure to store a sample of short DNA sequences (“Kmers”) with corresponding melting temperatures (“Tm”);
(c) profiling each Kmer for genomic abundance to identify candidates;
(d) profiling the candidates for a potential to mis-prime;
(e) profiling the candidates for sequence diversity in downstream flank;
(f) profiling the candidates for genomic uniformity;
(g) profiling the candidates for levels of selection;
(h) collapsing similar candidates using degenerate bases;
(i) evaluating alignments of flanking regions of the candidates;
(j) evaluating the potential for the candidates to be a suitable primer; and
(k) selecting at least one suitable repeat motif for use in subsequent steps in the method.

8. The method of claim 1, wherein the nucleic acid comprises DNA.

9. The method of claim 1, wherein said adapter primer includes a sequence that is at least partially homologous to the adapter sequence.

10. A method for simultaneously evaluating genomic variation in first and second species, the method comprising:
pooling (a) a first species nucleic acid from the first species, the first species nucleic acid having a first repeat motif and (b) a second species nucleic acid from the second species, the second species nucleic acid having a second repeat motif;
generating nucleic acid fragments by fragmenting the first species nucleic acid and the second species nucleic acid;
ligating an adapter molecule having an adapter sequence to at least one of the nucleic acid fragments; and
amplifying at least a portion of the nucleic acid fragments using a first tailed primer, a second tailed primer, and an adapter primer, the first tailed primer including a first nucleic acid sequence that binds to the first repeat motif and a second nucleic acid sequence that does not bind to at least one of said nucleic acid fragments having the first repeat motif, the second tailed primer including a third nucleic acid sequence that binds to the second repeat motif and a fourth nucleic acid sequence that does not bind to at least one of said nucleic acid fragments having the second repeat motif, the adapter primer including a sequence homologous to the adapter sequence, thereby producing amplified nucleic acid fragments.

11. The method of claim 10, wherein at least one of the first and second repeat motifs includes a nucleotide sequence including at least one of GTⁿ, GTⁿ-H, GTⁿ-HV, GTⁿ-A, V-GTⁿ, HV-GTⁿ, V-GTⁿ-H, HV-GTⁿ-HV, TGⁿ, ACⁿ, CAⁿ, and a reverse complement thereof.

12. The method of claim 10, wherein the first nucleic acid sequence is complementary to the first repeat motif and the third nucleic acid sequence is complementary to the second repeat motif.

13. The method of claim 12, wherein the second nucleic acid sequence is non-complementary to the at least one of said nucleic acid fragments having the first repeat motif, and the fourth nucleic acid sequence is non-complementary to the at least one of said nucleic acid fragments having the second repeat motif.

14. The method of claim 10, wherein the fragmenting comprises sonicating the first species nucleic acid and the second species nucleic acid.

15. The method of claim 10, wherein said adapter primer includes a sequence that is at least partially homologous to the adapter sequence.

16. A system for evaluating genomic variation in a nucleic acid fragment having a repeat motif, the system comprising:
a tailed primer including a first nucleic acid sequence that binds to the repeat motif and a second nucleic acid sequence that does not bind to the nucleic acid fragment.

17. The system of claim 16, further comprising an adapter primer having a sequence at least partially homologous to an adapter sequence at an end of the nucleic acid fragment.

18. The system of claim 16, wherein the nucleic acid comprises DNA.

19. The system of claim 16, wherein the repeat motif includes a nucleotide sequence including at least one of GTⁿ, GTⁿ-H, GTⁿ-HV, GTⁿ-A, V-GTⁿ, HV-GTⁿ, V-GTⁿ-H, HV-GTⁿ-HV, TGⁿ, ACⁿ, CAⁿ, and a reverse complement thereof.

20. The system of claim 17, wherein the second nucleic acid sequence comprises a P5 adapter sequence and the adapter sequence comprises a P7 adapter sequence.

21. The system of claim 16, further comprising a second tailed primer including a first nucleic acid sequence homologous to the repeat motif and a third nucleic acid sequence that is at least partially non-complementary to the nucleic acid fragment.

22. The system of claim 16, wherein the nucleic acid fragment comprises multiple nucleic acid fragments from divergent species.
